(19) United States
(12) Patent Application Publication
(10) Pub. No.: US 2024/0295134 A1
(43) Pub. Date: Sep. 5, 2024

(54) UTILITY POLE AND OXYGEN GENERATION METHOD USING IT

(71) Applicant: NIPPON TELEGRAPH AND TELEPHONE CORPORATION, Tokyo (JP)

(72) Inventors: Tatsuya MOKI, Musashino-shi, Tokyo (JP); Kenji INOUE, Musashino-shi, Tokyo (JP); Hidenobu HIROTA, Musashino-shi, Tokyo (JP); Yoshiyasu SATO, Musashino-shi, Tokyo (JP); Wataru KOKUBO, Musashino-shi, Tokyo (JP)

(73) Assignee: NIPPON TELEGRAPH AND TELEPHONE CORPORATION, Tokyo (JP)

(21) Appl. No.: 18/574,449

(22) PCT Filed: Jul. 5, 2021
(86) PCT No.: PCT/JP2021/025279

§ 371 Date: Dec. 27, 2023

(51) Int. Cl. E04H 12/02; C12P 3/00

(52) U.S. Cl. CPC E04H 12/02; C12P 3/00

(57) ABSTRACT

The present disclosure aims to provide a utility pole capable of generating oxygen, and an oxygen generation method using the utility pole.

The present disclosure relates to a utility pole that is formed with a transparent material that transmits sunlight, the utility pole having a cylindrical trapezoidal shape with a thin upper portion and a thick lower portion, an upper portion of the internal space of the utility pole being open, a lower portion of the internal space being closed.

### TECHNICAL FIELD

The present disclosure relates to a utility pole, and an oxygen generation method using the utility pole.

### BACKGROUND ART

Global warming is causing the temperature rise and the climate change that greatly change the climate of the entire earth. For example, it is recognized that people's lives are being damaged by large-scale typhoons that have been inconceivable before, or droughts that are caused because there are no rainfalls.

The increase in greenhouse gases such as carbon dioxide, methane, and chlorofluorocarbons in the atmosphere is cited as a factor of the climate change. It is known that these gases have increased since the industrial revolution in the 18th century. This is because human beings have burned fuels such as wood, coal, and oil to obtain large quantities of energy.

Carbon dioxide accounts for 70% of the greenhouse gases. Not only the generation of carbon dioxide has increased, but also the area of forests that absorb carbon dioxide has decreased. Plants in forests photosynthesize. Photosynthesis is synthesis of oxygen and organic substances (such as sugars) from carbon dioxide and water. The chemical formula is shown below in Formula (1). In formula (1), H2O represents water, CO2 represents carbon dioxide, O2 represents oxygen, and C6H12O6 represents an organic substance (saccharide).

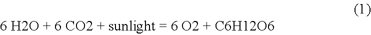

The rate of decrease in the forest area in the whole world is said to be equivalent to about two Tokyo Dome stadiums per minute. The decrease in the forest area means that the ability to produce oxygen from carbon dioxide is dropping.

Meanwhile, a large number of utility poles have been built in Japan. They are built along a track (FIG. 1) to supply power to trains running on the railway, and on a road (FIG. 2) to provide power and communication to customers' homes. In FIGS. 1 and 2, reference numeral 10 indicates a utility pole, reference numeral 21 indicates a communication cable, and reference numeral 22 denotes a power cable.

A structure of a standard utility pole that is currently adopted is now described. The materials include concrete and a steel material. Since a utility pole is to be built outdoors, the same materials as those of a structure such as a building are used. The steel material is formed into a columnar shape, and concrete is poured into the steel material while centrifugal separation is performed thereon, so that the center of the utility pole becomes hollow (see Non Patent Literatures 1 and 2, for example). FIG. 3 shows a cross-section perpendicular to the long axis direction of a utility pole. In FIG. 3, reference numeral 11 indicates a steel wire, reference numeral 12 indicates concrete, and reference numeral 13 indicates the internal space. The periphery of the steel wire 11 is covered with the concrete 12, and the central portion is hollow.

### CITATION LIST

### Non Patent Literature

Non Patent Literature 1: Homepage of Dainichi Concrete Industry Co., Ltd. http://www.dainichi-x.co.jp/service/pole/Non
Patent Literature 2: “Studies on the influence of environmental conditions on aged degradation of prestressed concrete poles”, Proceedings of the JCI, pp. 381-1386, Vol. 36, No. 2, 2014

### SUMMARY OF INVENTION

### Technical Problem

The number of utility poles built in Japan is 30 million or larger. Furthermore, about 50,000 to 100,000 utility poles are being newly built every year. Here, attention is drawn to the fact that this huge number of utility poles are located not indoors but outdoors. If a utility pole has a function of generating oxygen, the utility pole can be regarded as a tree. Since utility poles are located everywhere in town, there is a possibility that each town will become a forest that produces oxygen.

Therefore, to solve the above problem, the present disclosure aims to provide a utility pole capable of generating oxygen, and an oxygen generation method using the utility pole.

### Solution to Problem

To solve the above problem, a utility pole is formed with a material transparent to sunlight, and has a structure capable of accumulating water in an internal space.

Specifically, the present disclosure relates to
a utility pole that is formed with a transparent material that transmits sunlight,
the utility pole having a cylindrical trapezoidal shape with a thin upper portion and a thick lower portion,
an upper portion of the internal space of the utility pole being open,
a lower portion of the internal space being closed.

In the utility pole according to the present disclosure,
the transparent material may be plastic or glass.

In the utility pole according to the present disclosure,
a funnel having an upper portion thicker than the diameter of the internal space and a lower portion guided to the internal space is provided on the open upper portion.

To solve the above problem, water and algae are accumulated in the internal space of the utility pole to generate oxygen.

Specifically, the present disclosure relates to
an oxygen generation method that includes:
accumulating water and algae in the internal space of the utility pole described above; and irradiating the algae with sunlight, to cause the algae to generate oxygen.

### Advantageous Effects of Invention

According to the present disclosure, it is possible to provide a utility pole capable of generating oxygen. It is also possible to provide a method for efficiently generating oxygen.

### BRIEF DESCRIPTION OF DRAWINGS

FIG. 1 is a photograph of utility poles.

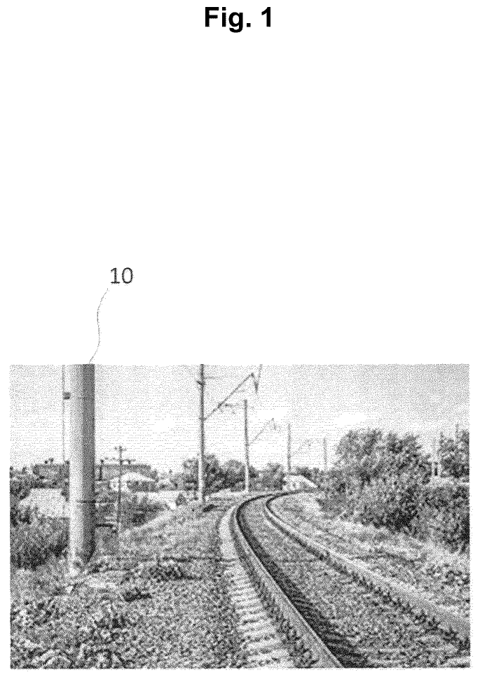

FIG. 2 is a photograph of a utility pole.

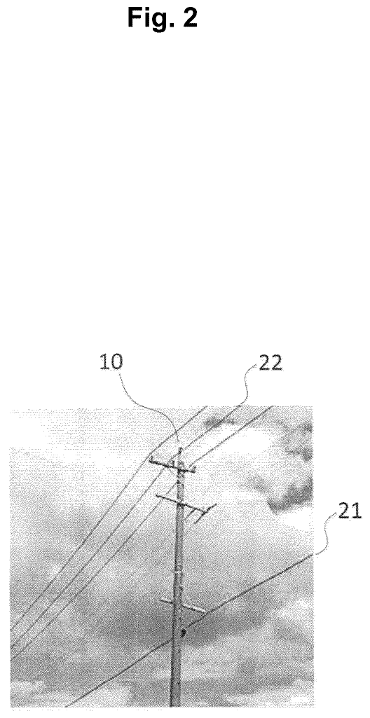

FIG. 3 is a cross-sectional view perpendicular to the long axis direction of a utility pole.

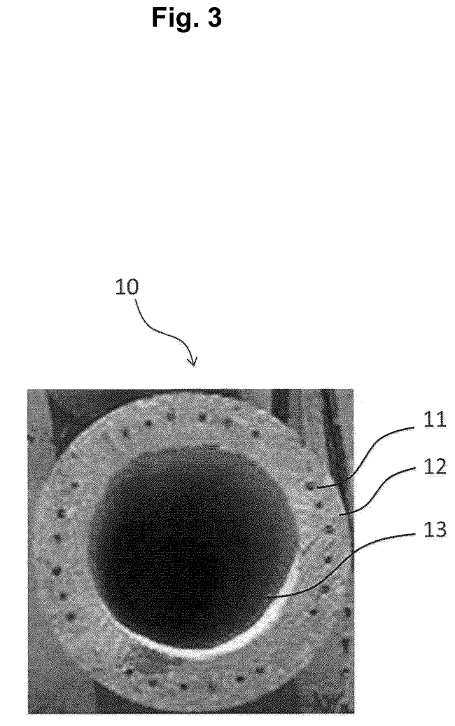

FIG. 4 is a diagram for roughly explaining the structure of a utility pole according to the present disclosure as viewed from a side.

FIG. 5 is a diagram for roughly explaining the structure of a utility pole according to the present disclosure as viewed from a side.

FIG. 6 is a diagram for explaining an oxygen generation method using a utility pole according to the present disclosure.

FIG. 7 is a diagram for explaining an oxygen generation method using a utility pole according to the present disclosure.

FIG. 8 is a diagram for explaining an oxygen generation method using a utility pole according to the present disclosure.

FIG. 9 is a diagram for illustrating a situation in which a utility pole according to the present disclosure is installed on the ground.

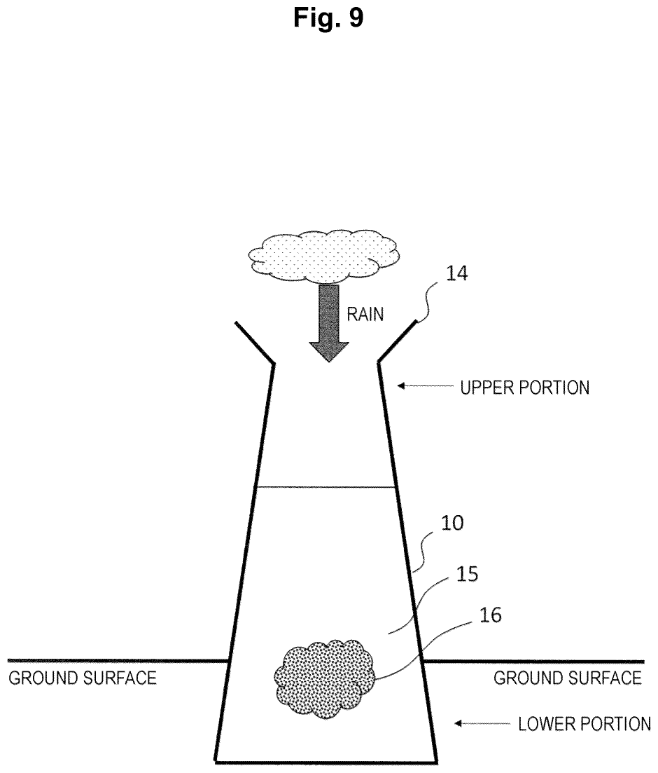

### DESCRIPTION OF EMBODIMENTS

The following is a detailed description of embodiments of the present disclosure, with reference to the drawings. Note that the present disclosure is not limited to the embodiments described below. These embodiments are merely examples, and the present disclosure can be carried out in forms with various modifications and improvements based on the knowledge of those skilled in the art. Note that components denoted by the same reference sign in the present specification and the drawings are the same.

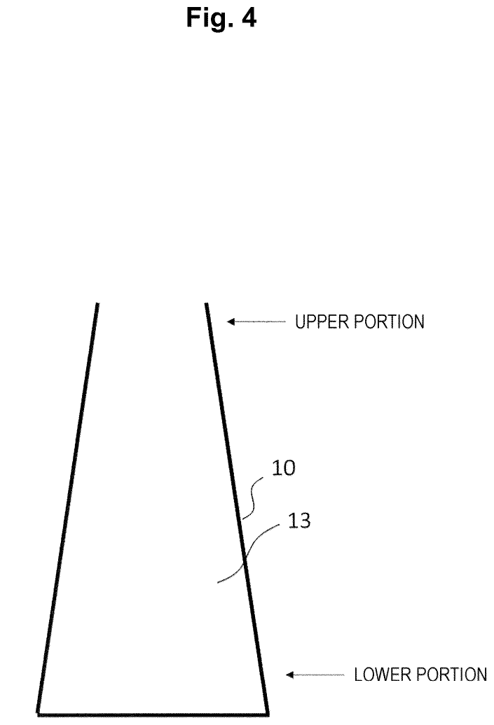

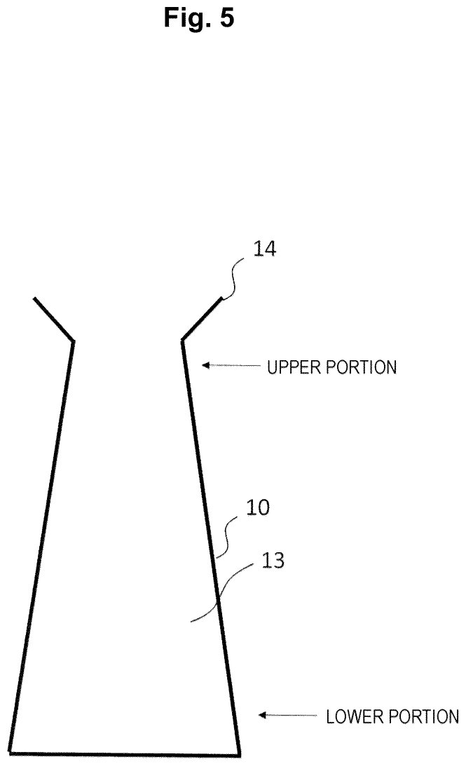

FIGS. 4 and 5 each schematically illustrates the structure of a utility pole according to the present disclosure as viewed from a side. In FIGS. 4 and 5, reference numeral 10 indicates a utility pole, reference numeral 13 indicates an internal space, and reference numeral 14 indicates a funnel. As illustrated in FIG. 4, the utility pole 10 has a tapered cylindrical trapezoidal shape with a thin upper portion and a thick lower portion. The utility pole according to the present disclosure is formed with a transparent material that transmits sunlight, to introduce sunlight into the internal space. Examples of the transparent material include transparent plastic and transparent glass. The upper portion of the internal space 13 is open, and the lower portion of the internal space 13 is closed. With this configuration, water can be supplied into and accumulated in the internal space 13 of the utility pole.

As illustrated in FIG. 5, a funnel 14 may be provided in the open upper portion of the internal space 13. The funnel 14 has a tapered shape that is the opposite of that of the utility pole. The upper portion of the funnel 14 is thicker than the diameter of the upper portion of the internal space 13, and the lower portion is guided to the upper portion of the internal space 13. With this structure, rain can be efficiently collected.

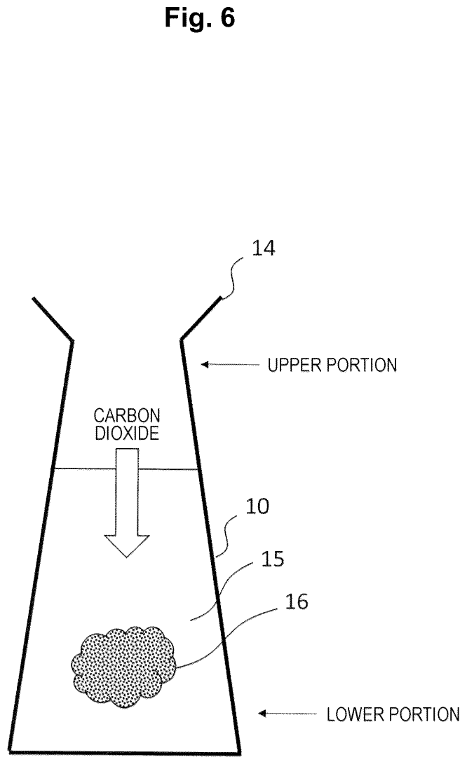

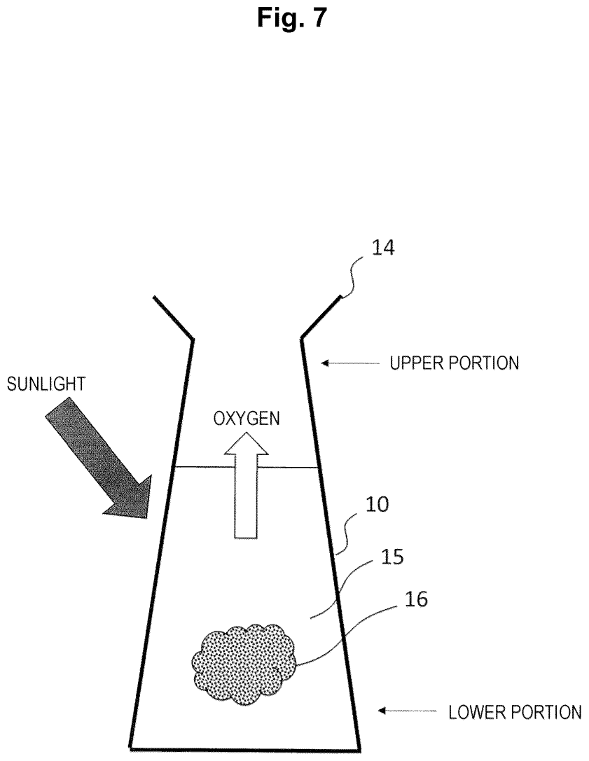

Referring to FIGS. 6, 7, and 8, an oxygen generation method using the utility pole according to the present disclosure is described, with the utility pole in FIG. 5 being taken as an example. This oxygen generation method can also be applied to a utility pole having the structure illustrated in FIG. 4. The same applies to the embodiment described below. In FIGS. 6, 7, and 8, reference numeral 10 indicates a utility pole, reference numeral 13 indicates an internal space, reference numeral 14 indicates a funnel, reference numeral 15 indicates water, and reference numeral 16 indicates algae. In FIG. 6, the water 15 is accumulated in the internal space 13 of the utility pole 10. Further, the algae 16 are also mixed in the water 15. Carbon dioxide dissolves into the water 15 from the atmosphere. In FIG. 7, when sunlight passes through the utility pole formed with a transparent material, and the algae 16 are irradiated with sunlight, oxygen is generated by the chemical reaction according to Formula (1) described above. Oxygen is released from the upper portion of the open internal space 13 to the outside of the utility pole.

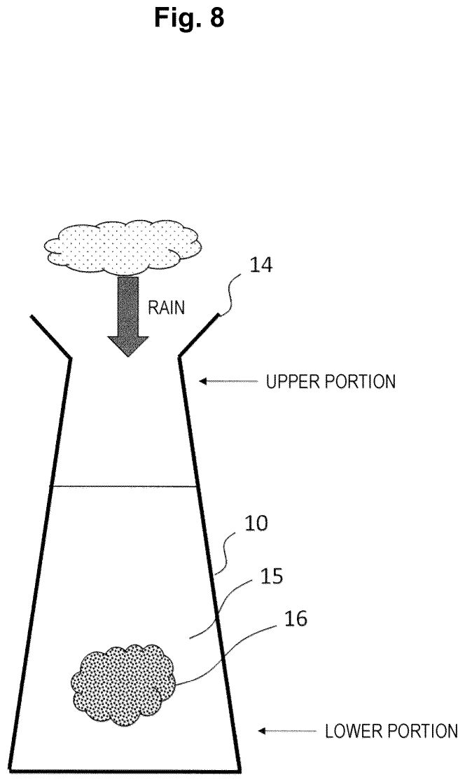

Water is necessary in the internal space 13 of the utility pole, to take advantage of the algae and generate oxygen. Water needs to be replenished from the outside of the utility pole, because moisture escapes into the atmosphere when evaporating. FIG. 8 illustrates water that is supplied to continue to generate oxygen. As the upper portion of the utility pole is open, rainwater can be collected inside the utility pole when it rains. In particular, as the funnel having a wider upper portion is provided on the upper portion of the utility pole, rainwater can be efficiently collected.

A conventional utility pole is formed with concrete and a steel material, and weighs one ton or more. On the other hand, a utility pole according to the present disclosure is formed with transparent plastic or glass, and accordingly, is considered to be lighter in weight than a conventional utility pole. In a case where a utility pole is light in weight, the utility pole might tilt when strong wind blows outside. In a utility pole according to the present disclosure, accumulation of water in a lower portion of the utility pole increases the weight of the utility pole, and thus, tilting due to strong wind is prevented.

As described so far, a utility pole according to the present disclosure can generate oxygen. Furthermore, when the utility pole according to the present disclosure is used, a method for efficiently generating oxygen can be provided.

### INDUSTRIAL APPLICABILITY

The present disclosure can be applied to communications industries.

### REFERENCE SIGNS LIST

10 utility pole
11 steel wire
12 concrete
13 internal space
14 funnel
15 water
16 algae
21 communication cable
22 power cable

1. A utility pole that is formed with a transparent material that transmits sunlight,
the utility pole having a cylindrical trapezoidal shape with a thin upper portion and a thick lower portion,
an upper portion of an internal space of the utility pole being open,
a lower portion of the internal space being closed.

2. The utility pole according to claim 1, wherein the transparent material is plastic or glass.

3. The utility pole according to claim 1, wherein a funnel having an upper portion thicker than a diameter of the internal space and a lower portion guided to the internal space is provided on the open upper portion.

4. An oxygen generation method comprising: accumulating water and algae in the internal space of the utility pole according to claim 1; and irradiating the algae with sunlight, to cause the algae to generate oxygen.
